(19) United States
(12) Patent Application Publication
(10) Pub. No.: US 2011/0046154 A1
(43) Pub. Date: Feb. 24, 2011

(54) USE OF AMINOPEPTIDASE INHIBITORS OR AZAINDOLE COMPOUNDS FOR PREVENTING OR TREATING CANCEROUS METASTASES FROM EPITHELIAL ORIGIN

(75) Inventors: Pierre Roux, Saint-Gely-Du-Fesc (FR); Marion De Toledo, Montpellier (FR); Jean-Paul Leonetti, Catelau-Le -Lez (FR); Christelle Anguille, Montpellier (FR)

Correspondence Address: OLIFF & BERRIDGE, PLC, P.O. BOX 320850, ALEXANDRIA, VA, 22320-4850, US

(73) Assignee: CENTRE NATIONAL DE LA RECHERCHE SCIENTIFIQUE, Paris (FR)

(21) Appl. No.: 12/812,501

(22) PCT Filed: Jan. 21, 2009
(86) PCT No.: PCT/FR2009/050081

§ 371 Date: Oct. 4, 2010

(30) Foreign Application Priority Data
Jan. 22, 2008 (FR) 0850389

(51) Int. Cl. A61K 31/497; A61K 31/517; A61K 31/136; A61K 31/505; A61K 31/44; A61K 31/4164; A61K 31/426; A61P 35/00

(52) U.S. Cl. 514/254.03; 514/266.31; 514/646; 514/274; 514/355; 514/391; 514/365

(57) ABSTRACT

The invention relates to the use of a compound selected from an aminopeptidase-inhibiting compound and an azaindole compound for producing a drug for preventing or treating cancerous metastases in humans or animals.

### FIELD OF THE INVENTION

The present invention relates to the field of the therapeutic treatment of cancers, and more particularly to the prevention or treatment of metastases, in the case of cancer in humans or animals.

### PRIOR ART

Generally speaking, around 35% of patients newly diagnosed as suffering from a cancer are free from metastases. These patients can be cured by local treatments directed against the tumor, such as surgery or exposure to certain types of radiation.

The remainder of the newly diagnosed patients includes (i) patients already having detectable metastases (30% of the patients) and (ii) patients having no detectable metastases at the time of diagnosis (35% of the patients). The treatment of these remaining patients is generally a systemic treatment including, for example, the administration of chemotherapeutic drugs which interfere with the growth of the cancerous cells.

The processes of tumoral invasion constitute the essential cause of mortality in patients suffering from a cancer.

It is known that, in certain cancers, the tumor cells not only have a high capacity to proliferate but also have a high capacity to destroy tissues and to infiltrate into neighboring tissues, including the blood vessels and the lymphatic vessels, and then in migrating through the blood or lymphatic circulation to localized tissues at sites in the body that are very far away from the primary tumor. Accordingly, in certain cancers, the tumoral cells have a capacity to circulate and then to form secondary tumors, also called metastases, in tissues which are distant from the primary tumor.

The formation of metastases is a multi-stage physiological phenomenon in the course of which the tumoral cells detach from the primary tumor, invade the extracellular matrix, penetrate through blood vessels, enter into the vascular system by intravasation, then halt their migration in the blood or lymphatic circulation at a distant site, emerge from the circulation by extravasation, and then become fixed in a distant tissue and proliferate to form a secondary tumor.

Furthermore, the epithelium represents the tissue type which is most widespread within the human or animal body. This is doubtless one of the reasons why more than 90% of cancers in humans originate in the malignant transformation of epithelial cells. In particular, the epithelial cancer of colorectal type constitutes the second cause of death by cancer in industrialized countries.

In the majority of cancer cases, including those involving epithelial cancers, the mortality is due not to the primary tumor but to the metastases which spread systemically within the body, from the primary tumor. This malignant progression, which leads to tumoral invasion, which is manifested clinically in the generation of metastases, is conditioned initially by the loss of cellular adhesion on the part of certain tumoral cells and also by an increase in cell motility, which means that these invasive tumoral cells depart the tissue of the primary tumor and then colonize one or more target tissues, which in some cases are very distant from the site of the primary tumor, in the human or animal body. In epithelial cancers, the malignant progression is accompanied by a loss of the intercellular junctions induced by E-cadherin, at the primary tumor.

Among epithelial cancers, colorectal cancers represent the second-most frequent cancers in industrialized countries. The evolution of colorectal cancer is a succession of events that begins with an initiation phase due to a genomic deregulation impairing the proliferative properties of the cells. The second phase is a phase of tumoral progression, leading to changes in cell morphology and to the emergence of transformed cell clones. The final phase, lastly, comprises the phase of invasion consecutive to epithelial-mesenchymal transition, a transition defined as the capacity of an epithelial cell to separate from neighboring cells and to migrate toward other sites in the body.

It has been shown, in practice, that systemic therapeutic anticancer treatments have little effect on metastases, and especially on macro-metastases, which are resident in organs distant from the site of the primary tumor, such as the lung, liver, bone marrow or brain. Cancer patients therefore often die because of metastatic cancers provoked by the metastases of the cancerous cells.

Furthermore, to the present date, research into anti-invasive molecules has generally been based either (i) on the capacity of the candidate compounds to inhibit the growth of tumoral cells or (ii) on the aptitude of the candidate compounds for inhibiting the migration of the cells. According to the first strategy, however, the compounds selected have no effect on the spread of invasive cells and hence no effect on the metastatic process itself. And, in the second case, the compounds may have an inhibitory activity on the capacity of the cells to migrate in the body and to form metastases, but have no effect on metastatic cells which have already migrated.

There is therefore a need within the art for the identification of active substances which could possess the capacity to prevent or treat metastases in patients suffering from a cancer, so as to increase significantly the chances of a cure in these patients.

### SUMMARY OF THE INVENTION

The present invention provides for the use of a compound selected from an aminopeptidase inhibitor compound and an azaindole compound for the manufacture of a medicament intended for the prevention or treatment of cancerous metastases in humans or animals.

The present invention relates more particularly to the use, for the manufacture of a medicament intended for the prevention or treatment of cancerous metastases in humans or animals, of an antimetastatic compound selected from the compounds of formula (I), (II), (III), (IV), (V) and (VI), which are described in detail later on in the present description.

The invention pertains to the use defined above for the prevention or treatment of metastases in the case of epithelial cancers, including in the case of colorectal cancers and breast, liver, pancreatic, prostate, and uterine cancers.

### DESCRIPTION OF THE FIGURES

FIG. 1 is a diagram illustrating the antimetastatic effect of each of the compounds of formulae (I), (II), (III), (IV), (V) and (VI) of the invention. On the ordinate: the invasion capacity of the cells tested, as a percentage relative to the invasion capacity of cells from the line SW620 (ATCC No. CCL-227), which was fixed arbitrarily at 100%. On the abscissa, from left to right: (i) cells of the colorectal metastatic line SW620, incubated with the culture medium only; (ii) cells of the nonmetastatic line Hct116, incubated with the medium only; and (iii) cells of the metastatic line SW620, incubated with the culture medium with addition of each of the compounds of formula (I), (II), (III), (IV), (V) and (VI). The results shown in FIG. 1 are an average from three experiments.

FIG. 2 is a diagram illustrating the antimetastatic effect of each of the compounds of formulae (I), (II), (III), (IV), (V) and (VI) of the invention in other cell lines. On the ordinate: the invasion capacity of the cells tested, as a percentage relative to the invasion capacity of cells of the line CoLo205 (ATCC No. CCL-222), which was fixed arbitrarily at 100%. On the abscissa, from left to right: (i) cells of the colorectal metastatic line CoLo205 (ATCC No. CCL-222), incubated with the culture medium alone or with addition of each of the compounds of formula (I), (II), (III), (IV), (V) and (VI). The results shown in FIG. 2 are an average from three experiments.

FIG. 3 is a diagram illustrating the antimetastatic effect of each of the compounds of formulae (I), (II), (III), (IV), (V) and (VI) of the invention. On the ordinate: the invasion capacity of the cells tested, as a percentage relative to the invasion capacity of cells from the colorectal metastatic line SK-Col (ATCC No. HTB-77), which was fixed arbitrarily at 100%. On the abscissa, from left to right: (i) cells of the colorectal metastatic line SK-Col (ATCC No. HTB-77), incubated with the culture medium only or with addition of each of the compounds of formula (I), (II), (III), (IV), (V) and (VI). The results shown in FIG. 3 are an average from three experiments.

FIG. 4 is a diagram illustrating the antimetastatic effect of each of the compounds of formulae (I), (II), (III), (IV), (V) and (VI) of the invention. On the ordinate: the invasion capacity of the cells tested, as a percentage relative to the invasion capacity of cells of the mammary adenocarcinoma metastatic line MDA-MB-231 (ATCC No. HTB-26), which was fixed arbitrarily at 100%. On the abscissa, from left to right: (i) cells of the metastatic line SW620, incubated with the culture medium only; (ii) cells of the nonmetastatic line Hct116, incubated with the medium only; and (iii) metastatic cells of the mammary adenocarcinoma metastatic line MDA-MB-231 (ATCC No. HTB-26), incubated with the culture medium alone or with addition of each of the compounds of formula (I), (II), (III), (IV), (V) and (VI). The results shown in FIG. 4 are an average from three experiments.

### DETAILED DESCRIPTION OF THE INVENTION

The present invention provides new means for preventing or treating metastases in various cancers, especially in epithelial cancers, including in colorectal cancers, breast cancers, liver cancers, cancers of the pancreas, cancers of the prostate, and cancers of the uterus.

Surprisingly, it has been shown according to the invention that aminopeptidase inhibitor compounds and compounds from the class of the azaindoles have the capacity to inhibit or block the invasive properties of cancerous cells initially able to generate metastases.

More specifically, it is shown according to the invention that aminopeptidase inhibitor compounds and compounds from the class of the azaindoles have the capacity to inhibit or block the migration properties of metastatic cells having their origin in various epithelial cancers, such as colorectal cancers or else breast cancers.

The identification of the antimetastatic properties of these compounds has allowed the applicant to develop pharmaceutical compositions for preventing or treating cancerous metastases in humans or animals, said compositions comprising, as active principle, at least one compound selected from an aminopeptidase inhibitor compound and an azaindole compound. This identification of new therapeutic or preventive properties has enabled the use of these compounds for the manufacture of pharmaceutical compositions having antimetastatic activity.

The present invention accordingly provides a compound selected from an aminopeptidase inhibitor compound and an azaindole compound for preventing or treating cancerous metastases in humans or animals.

The present invention further provides for the use of a compound selected from an aminopeptidase inhibitor compound and an azaindole compound for manufacturing a medicament intended for the prevention or treatment of cancerous metastases in humans or animals.

As illustrated in the examples, the aminopeptidase inhibitor compounds and the azaindole compounds identified according to the invention have the capacity to induce a reversion of the invasive phenotype of cancerous cells such as metastatic colorectal carcinoma cells and metastatic mammary adenocarcinoma cells.

Moreover, the applicant has shown that the aminopeptidase inhibitor compounds and the azaindole compounds identified according to the invention are not cytotoxic.

Without wishing to be bound by any one theory, the applicant considers that the antimetastatic properties of the aminopeptidase inhibitor compounds and azaindole compounds identified according to the invention are due to the capacity, common to the totality of these compounds, to induce the re-establishment of the cellular junctions associated with the expression of E-cadherin at the surface of the metastatic cancerous cells.

The capacity of the antimetastatic compounds of the invention to induce the expression of E-cadherin at the surface of cancerous cells and hence to induce the re-establishment of the cellular junctions associated with this expression of E-cadherin may be verified, in particular, by performing the test described in the PCT application published under no. WO 2006/134305 (Centre National de la Recherche Scientifique—Pierre Roux and Marion De Toledo).

According to a first embodiment of an antimetastatic compound which is used according to the invention, said compound consists of an aminopeptidase inhibitor compound of formula (I) below:

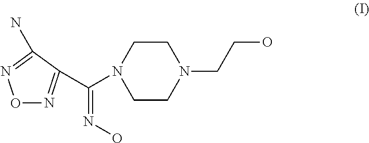

### Structural Analogs of the Compound (I) to be Specified by the Inventors, Please

The compound of formula (I) is also denoted in the present description as the compound 2-(4-[(4-amino-1,2,5-oxadiazol-3-yl)(hydroxyimino)methyl]-1-piperazinyl)ethanol.

The compounds of formula (I) can be synthesized by any synthesis process known to a person skilled in the art. In order to synthesize a compound of formula (I), a person skilled in the art may in particular refer to the process described in the following document: Synthesis of secondary and tertiary aminofurazans. Sheremetev, A. B.; Andrianov, V. G.; Mantseva, E. V.; Shatunova, E. V.; Aleksandrova, N. S.; Yudin, I. L.; Dmitriev, D. E.; Averkiev, B. B.; Antipin, M. Yu. N. D. Zelinsky Institute of Organic Chemistry, Russian Academy of Sciences, Moscow, Russia. Russian Chemical Bulletin (Translation of Izvestiya Akademii Nauk, Seriya Khimicheskaya) (2004), 53(3), 596-614. Publisher: Kluwer Academic/Consultants Bureau, CODEN: RCBUEY ISSN: 1066-5285. Journal written in English. CAN 142:219211 AN 2004: 589877 CAPLUS.

According to a second embodiment of an antimetastatic compound which is used according to the invention, said compound consists of an azaindole compound of formula (II) below:

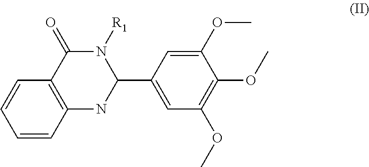

in which the group R₁ is a linear or branched alkyl having from 1 to 8 carbon atoms.

By “alkyl” is meant a linear or branched aliphatic hydrocarbon group which is optionally interrupted by a heteroatom, it being possible for the alkyl to be unsubstituted or substituted on the carbon atoms by one or more identical or different substituents, the substituents being selected from: aryl, hydroxyl, alkoxy, aryloxy, alkyloxy, aralkyloxy. By “branched” alkyl is meant a lower alkyl having 1, 2, 3, 4 or 5 carbon atoms, such as methyl, ethyl or propyl, which is bonded to a linear alkyl chain. Preferred alkyl groups consist of alkyl groups having 1, 2, 3, 4, 5, 6, 7 or 8 carbon atoms. Examples of alkyl groups are methyl, ethyl, isopropyl, tert-butyl, heptyl, decyl or cyclohexylmethyl.

By “alkoxy” is meant an alkyl-O— group in which the alkyl group is as defined above. Alkoxy groups include methoxy, ethoxy, n-propoxy, isopropoxy, n-butoxy, and heptoxy.

By “aryloxy” is meant an aryl-O group in which the aryl group is as defined above. Aryloxy groups include phenoxy and naphthoxy.

By “aralkyl” is meant an alkyl group substituted by one or more aryl groups.

By “aralkyloxy” is meant an aralkyl-O— group in which the aralkyl group is as defined above. Aralkyloxy groups include benzyloxy.

One preferred compound of formula (II) according to the invention is the compound in which the group R₁ signifies a propyl group, and may also be denoted in the present description as the compound methyl 4-[([3-(4-fluorophenyl)-4-oxo-2-thioxo-1,3-thiazolidin-5-ylidene)methyl)benzoate.

The compounds of formula (II) may be synthesized by any synthesis process known to a person skilled in the art. In order to synthesize a compound of formula (II), a person skilled in the art may in particular refer to the process described in the following document: quinazolinone compounds. (Tanabe Seiyaku Co., Ltd., Japan). Jpn. Kokai Tokkyo Koho (1982), 5 pp. CODEN: JKXXAF JP 57011970 A 19820121 Showa. Patent written in Japanese. Application: JP 80-86044 19800624. Priority: CAN 97:72383 AN 1982:472383 CAPLUS. According to a third embodiment of an antimetastatic compound which is used according to the invention, said compound consists of an azaindole compound of formula (III) below:

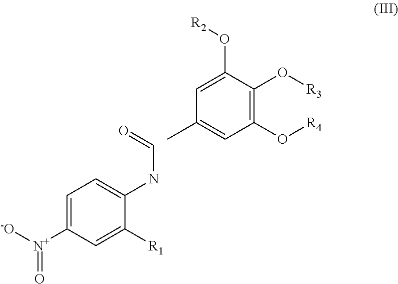

in which the groups R₁, R₂, R₃, and R₄ each represent, independently of one another, an alkyl of 1 to 8 carbon atoms, whose definition is identical to that of the group R₁ in the compound of formula (II).

One preferred compound of formula (III) is the compound in which the group R₁ signifies a methyl group and the groups R₂, R₃, and R₄ signify an ethyl group, it also being possible for said compound to be denoted as the compound 3,4,5-triethoxy-N-(2-methyl-4-nitrophenyl)-benzamide.

The compounds of formula (III) may be synthesized by any synthesis process known to a person skilled in the art. In order to synthesize a compound of formula (III), a person skilled in the art may in particular refer to the process described in the following document: Microwave-assisted synthesis of salicylamide via BC13 mediated coupling. Zhang, Lei; Zhang, John Y. CytRx Laboratories, Inc., Worcester, Mass., USA. Journal of Combinatorial Chemistry (2005), 7(4), 622-626. Publisher: American Chemical Society, CODEN: JCCHFF ISSN: 1520-4766. Journal written in English. CAN 143:211699 AN 2005:538799 CAPLUS. According to a fourth embodiment of an antimetastatic compound which is used according to the invention, said compound consists of an azaindole compound of formula (IV) below:

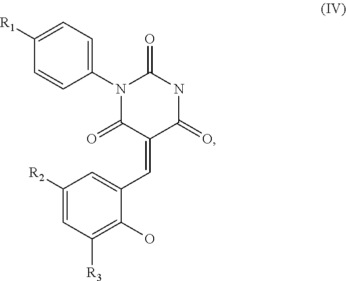

in which the groups R₁, R₂, and R₃ each represent, independently of one another, a halogen selected from a fluorine (F), chlorine (C)l, iodine (I), and bromine (B)r atom.

One preferred compound of formula (IV) according to the invention is the compound in which the groups R₁ and R³ each signify a chlorine atom and the group R₂ signifies a bromine atom, it also being possible for said compound to be denoted 5-(5-bromo-3-chloro-2-hydroxybenzylidene)-1-(4-chlorophenyl)-2,4,6-(1H,3H,5H)-pyrimidinetrione.

The compounds of formula (IV) may be synthesized by any synthesis process known to a person skilled in the art. In order to synthesize a compound of formula (IV), a person skilled in the art may in particular refer to the process described in the following document: Synthesis of pyrimidine derivatives possessing an antioxidative property and their inhibitory effects on picryl chloride-induced contact hypersensitivity reaction. Isobe, Yoshiaki; Hirota, Kosaku. Pharmaceuticals and Biotechnology Laboratory, Japan Energy Corporation, Saitama, Japan. Chemical & Pharmaceutical Bulletin (2003), 51(12), 1451-1454. Publisher: Pharmaceutical Society of Japan, CODEN: CPBTAL ISSN: 0009-2363. Journal written in English. CAN 140:174446 AN 2003:989349 CAPLUS.

According to a fifth embodiment of an antimetastatic compound which is used according to the invention, said compound consists of an azaindole compound of formula (V) below:

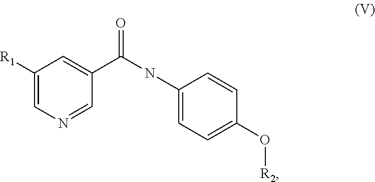

in which:
the group R₁ is a halogen selected from a fluorine (F), chlorine (C)l, iodine (I), and bromine (B)r atom, and
the group R₂ is a linear or branched alkyl having from 1 to 8 carbon atoms, whose definition is identical to that of the group R₁ in the compound of formula (II).

One preferred compound of formula (V) is the compound in which the group R1 signifies a bromine atom and the group R2 signifies an ethyl group, it also being possible for said compound to be denoted as the compound 5-bromo-N-(4-butoxyphenyl)nicotinamide.

The compounds of formula (V) may be synthesized by any synthesis process known to a person skilled in the art. In order to synthesize a compound of formula (V), a person skilled in the art may in particular refer to the process described in the following document: Application of organolithium and related reagents in synthesis, 30. Behavior of N-pyridylbenzamides versus benzanilides in the ortho-directed lithiation of masked aromatic carboxylic acids. Jozwiak, Andrzej; Brzezinski, Jacek Z.; Plotka, Mieczyslaw W.; Szczesniak, Aleksandra K.; Malinowski, Zbigniew; Epsztajn, Jan. Department of Organic Chemistry, Institute of Chemistry, University of Lodz, Lodz, Pol. European Journal of Organic Chemistry (2004), (15), 3254-3261. Publisher: Wiley-VCH Verlag GmbH & Co. KGaA, CODEN: EJOCFK ISSN: 1434-193X. Journal written in English. CAN141:295825 AN 2004:636845 CAPLUS.

According to a sixth embodiment of an antimetastatic compound which is used according to the invention, said compound consists of an azaindole compound of formula (VI) below:

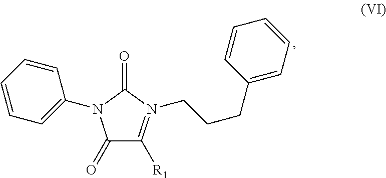

in which the group R₁ is a halogen selected from a fluorine (F), a chlorine (C)l, iodine (I), and bromine (B)r atom.

The compound of formula (VI) may also be designated the compound 3-chloro-1-phenyl-4-[(2-phenylethyl)amino]-1H-pyrrole-2,5-dione.

The compounds of formula (VI) may be synthesized by any synthesis process known to a person skilled in the art. The compounds of formula (VI) are compounds which are readily available commercially. Mention may be made in particular of the compound of formula VI which is 3-chloro-1-phenyl-4-[(2-phenylethyl)amino]-1H-pyrrole-2,5-dione, which is sold in particular by the company Chembridge (San Diego, United States of America) under the reference ID 6137235.

Thus, according to the invention, the preferred active principles which are used for manufacturing a medicament intended for the prevention or treatment of cancerous metastases in humans or animals are selected from:
methyl 4-[([3-(4-fluorophenyl)-4-oxo-2-thioxo-1,3-thiazolidin-5-ylidene)methyl)benzoate;
3,4,5-triethoxy-N-(2-methyl-4-nitrophenyl)benzamide;
5-(5-bromo-3-chloro-2-hydroxybenzylidene)-1-(4-chlorophenyl)-2,4,6-(1H,3H,5H)pyrimidinetrione;
5-bromo-N-(4-butoxyphenyl)nicotinamide; and
3-chloro-1-phenyl-4-[(2-phenylethyl)amino]-1H-pyrrole-2,5-dione.

According to a first preferred aspect, the aminopeptidase inhibitor compounds and the azaindole compounds of interest according to the invention are used for manufacturing a medicament intended for the prevention or for the treatment of epithelial cancers.

According to a second preferred aspect, the aminopeptidase inhibitor compounds and the azaindole compounds of interest according to the invention are used for manufacturing a medicament intended for the prevention or for the treatment of an epithelial cancer selected from a colorectal cancer and a breast cancer.

The pharmaceutical compositions according to the invention include more particularly those suitable for oral, parenteral, nasal, percutaneous, transcutaneous, rectal, perlingual or inhalative administration, and especially plain or coated tablets, sublingual tablets, gel capsules, wafers, suppositories, creams, ointments, dermal gels, and ingestible or injectable ampoules.

The dosage varies according to the sex, age and weight of the patient, according to the route of administration, and according to the type of cancer, the state of progression of the cancer, particularly according to whether metastases have or have not been detected in the patient. The dosage may also vary according to the type of any associated anticancer treatment or treatments.

Generally speaking, an antimetastatic compound as defined in the present description is used in amounts of from 0.01 mg to 1 g per 24 hours for an adult man or woman with an average weight of 80 kilos, in one or more doses.

A pharmaceutical composition according to the invention comprises the antimetastatic compound in combination with at least one excipient selected from the group consisting of pharmaceutically acceptable excipients.

Generally speaking, a pharmaceutical composition according to the invention comprises from 0.01% to 99% by weight, and advantageously from 1% to 90% by weight, of an antimetastatic compound, relative to the total weight of said composition.

Generally speaking, a pharmaceutical composition according to the invention comprises from 1% to 99.99% by weight, and advantageously from 10% to 99% by weight, of a pharmaceutically acceptable excipient or combination of such excipients.

The pharmaceutical composition of the present invention may be used for parenteral, topical or local administration and may be used preventively and/or therapeutically. Accordingly, the antimetastatic compound according to the present invention is prepared in a form suitable for the type of administration selected, as for example in liquid form or in lyophilized form. The pharmaceutical compositions comprising an antimetastatic compound according to the invention may contain an excipient and/or a vehicle which is pharmaceutically acceptable, preferably aqueous. Numerous pharmaceutically acceptable excipients and/or vehicles may be used, examples being water, buffered water, a saline solution, a solution of glycine and derivatives thereof, and also agents needed in order to reproduce physiological conditions, such as, for example, buffers and pH modifiers, surfactants such as sodium acetate, sodium lactate, sodium chloride, potassium chloride, calcium chloride, this list not being limitative. Moreover, the pharmaceutical composition may be sterilized by sterilization techniques that are well known to a person skilled in the art.

For the inert, nontoxic, and pharmaceutically acceptable excipients or vehicles, mention may also be made, by way of indication and not of limitation, of diluents, solvents, preservatives, wetting agents, emulsifiers, dispersants, binders, swelling agents, disintegrants, retardants, lubricants, absorbers, suspension agents, colorants, flavors, etc.

When a solid composition is prepared in the form of tablets, the principal active ingredient is mixed with a pharmaceutical vehicle such as gelatin, starch, lactose, magnesium stearate, talc, gum arabic or the like.

The tablets may be coated with sucrose or other appropriate starting materials or else may be treated such as to have prolonged or retarded activity and to release continuously a predetermined amount of active principle.

A preparation in the form of gel capsules is obtained by mixing the active ingredient with a diluent and pouring the resulting mixture into soft or hard gel capsules.

A pharmaceutical composition in the form of a syrup or elixir may comprise the active ingredient together with a sweetener, preferably a noncalorific sweetener, with methylparaben and propylparaben as antiseptics, and with an agent imparting taste and an appropriate colorant.

The powders or granules which are dispersible in water may comprise the active ingredient in a mixture with dispersants or wetting agents or suspension agents, such as polyvinylpyrrolodinone, and also with sweeteners or taste corrigents.

The active principle may also be formulated in the form of microcapsules, optionally with one or more carriers or additives.

Generally speaking, for the manufacture of a pharmaceutical composition in accordance with the invention, a person skilled in the art may advantageously refer to the most recent edition of the European Pharmacopeia, as for example to the 5th edition of the European Pharmacopeia, published January 2005, or else to the 6th edition of the European Pharmacopeia, available to the public in June 2007.

Techniques for preparing pharmaceutical compositions according to the invention can readily be found by a person skilled in the art, as for example in the work Remmingston's Pharmaceutical Sciences, Mid. Publishing Co., Easton, Pa., USA.

Physiologically acceptable adjuvants, vehicles, and excipients are also described in the work entitled “Handbook of Pharmaceutical Excipients”, second edition, American Pharmaceutical Association, 1994.

In order to formulate a pharmaceutical composition according to the invention, a person skilled in the art will be able advantageously to refer to the most recent edition of the European Pharmacopeia or of the Pharmacopeia of the United States of America (USP).

A person skilled in the art will be able in particular, advantageously, to refer to the USP 30-NF 25 edition of the American Pharmacopeia (U.S. Pharmacopeia).

Advantageously, a pharmaceutical composition as defined above is adapted for oral, parenteral or intravenous administration.

When the pharmaceutical composition according to the invention comprises at least one pharmaceutical or physiologically acceptable excipient, the excipient in question is more particularly an excipient appropriate for administration of the composition orally or an excipient appropriate for administration of the composition parenterally.

The invention also relates to a method of preventing or treating a disorder associated with an imbalance in bone metabolism, more particularly a disorder associated with a loss of bone mass, said method comprising a step in which the patients are administered a therapeutically effective amount of an antimetastatic compound as described in the present description or else of a pharmaceutical composition comprising said antimetastatic compound.

A pharmaceutical composition comprising an antimetastatic compound according to the invention may be present equally well in either a solid form or a liquid form.

For oral administration, preference will be given to a solid pharmaceutical composition, in the form of tablets, plain capsules or gel capsules.

In the liquid form, preference will be given to a pharmaceutical composition in the form of an aqueous suspension or nonaqueous suspension, or else in the form of a water-in-oil or oil-in-water emulsion.

Vehicles, adjuvants or excipients present in solid pharmaceutical forms may comprise at least one diluent, flavor, solubilizer, lubricant, suspension agent, binder, disintegrant, and encapsulant.

Such compounds are, for example, magnesium carbonate, magnesium stearate, talc, lactose, pectin, dextrin, starch, gelatin, cellulosic materials, cocoa butter, etc.

The compositions in liquid form may also comprise water, where appropriate as a mixture with propylene glycol or polyethylene glycol, and also, optionally, colorants, flavors, stabilizers, and thickeners.

The invention is further illustrated, but not limited, by the example below.

### Example 1

### Anti-Invasive Properties of the Antimetastatic Compounds of the Invention

### A. Equipment and Methods

### 1. E-cadherin Labeling Protocol in 96-Well Plates

1-Plate approximately 10 000 cells per well for colorectal lines (in Falcon 96-well plates, ref: 353072).
2-Carry out the necessary treatments (control, compound (I), (II), etc. for 48 h) and carry out fixing for 10 minutes with formalin (formaldehyde 3.7% in PBS).
3-Saturation, 10 minutes PBS-BSA.
4-Incubation of the primary antibody, diluted 1:500 in PBS-BSA, 2 hours at 37° C. (mouse anti-E-cadherin, Zymed, ref: 13-1700).
5-Two rapid rinses with PBS—0.1% Tween.
6-Incubation of biotinylated anti-mouse Ab, diluted 1:1000 in PBS-BSA, 30 minutes at 37° C. (anti-mouse IgG, heavy and light chain specific biotin conjugate, Calbiochem, ref: 401213).
7-Two rapid rinses with PBS—0.1% Tween.
8-Incubation of streptavidin-HRP, diluted between 1:1000 in PBS-BSA, 30 minutes at 37° C. (ECL streptavidin-horseradish peroxidase conjugate, Amersham Biosciences, ref: RPN1231).
9-Two rapid rinses with PBS—0.1% Tween.

### 10-Visualization:

Dissolve one capsule of Phosphate-citrate buffer containing sodium perborate (Sigma, ref: P4922) in 100 ml of deionized water. This buffer must be used within 30 minutes after its reconstitution.

Dissolve wafers of o-phenylenediamine (OPD) dihydrochloride (Sigma, ref: P6787) in this buffer to give a final concentration of 0.4 mg/ml (1 pellet of OPD at 10 mg in 40 ml of perborate buffer).

Introduce 100 μl of OPD/perborate per well and carry out incubation at ambient temperature for 5 minutes.

Stop the reaction with 50 μl of 3N HCl.

Read off the OD at 490 nm.

### 2. Invasion Test

1-On D−2, thaw the Matrigel at 4° C. overnight.
2-On D−1, pour the Matrigel into the chambers:

Place all of the required equipment on ice (matrigel, culture medium, tips, eppendorfs, etc.).

Introduce the Fluoroblock inserts (Falcon, ref: 351152) into 24-well plates (Falcon, ref: 353504).

Dilute the matrigel to 2 mg/ml in the cold serumless culture medium (if the cells are to undergo a treatment X, include the product in the matrigel, e.g.: Y27632).

Distribute 100 μl of 2 mg/ml matrigel per insert, avoiding formation of bubbles.

Leave O/N at 37° C. in a humid atmosphere.

3-On day D, plate the cells on the matrigel:

Firstly, introduce 700 μl of 10% serum medium into a 24-well plate, and transfer the inserts to this plate.

Trypsinate the cells (pretreated or otherwise) and take them up in 2% serum medium.

Seed 50 000 cells per insert in 200 μl of 2% serum medium onto the matrigel (where appropriate, place Y27632 in the plated cells).

4-The migration time is variable depending on cell type, but, generally, allowing migration for 8 hours, fixing a point every 2 hours, appears to be suitable.

To carry out fixing, transfer the inserts to a 24-well plate containing 1 ml of formalin (formaldehyde 3.7% in PBS), draw off the culture medium inside the chambers, and also add formalin (very important, preventing the cells from continuing to migrate within the matrigel). Incubate for 10 minutes.

Carry out 3 rapid rinses with PBS.

Subsequently, carry out labeling with propidium iodide by incubating the inserts in 1 ml of propidium iodide (Sigma, ref: P-4864), 1:500 in PBS, O/N at 4° C., in the dark.

### B. Results

### B.1. Capacity of the Antimetastatic Compounds of the Invention to Induce the Re-Establishment of Intercellular Junctions

The compounds of formula (I) to (VI) were tested for their capacity to bring about the re-establishment of intercellular junctions in in vitro cultures of cells of the line SW620, using the E-cadherin labeling test described in the Equipment and Methods section.

As a negative control, cells from the line SW620 were used, incubated in the culture medium alone, in the absence of antimetastatic compound. An average OD of 0.147 was obtained.

As a positive control, cells from the line HCT116 were used, incubated in the culture medium alone, in the absence of antimetastatic compound. An average OD of 0.734 was obtained.

The tests were carried out by incubating the cells from the line SW620 with each of the compounds (I) to (VI).

In these tests, the average OD obtained with each of the compounds (I) to (VI) was always greater than 0.300, which indicates that the compounds (I) to (VI) are capable of inducing cell spreading, the formation of compact cellular islets, and of bringing about the formation of new intercellular E-cadherin junctions.

### B.2. Anti-Invasive Properties of the Antimetastatic Compounds of the Invention

Measurements were made of the capacity of each of the compounds of formula (I) to (VI) to inhibit or block the invasive properties of metastatic cancerous cells, using the invasion test described in the Equipment and Methods section.

Cells from the metastatic line SW620, incubated in the absence of antimetastatic compound, were used as a negative control, for which the control value was set arbitrarily at 100%.

Cells from the nonmetastatic cancer line HCT116 were used as a positive control.

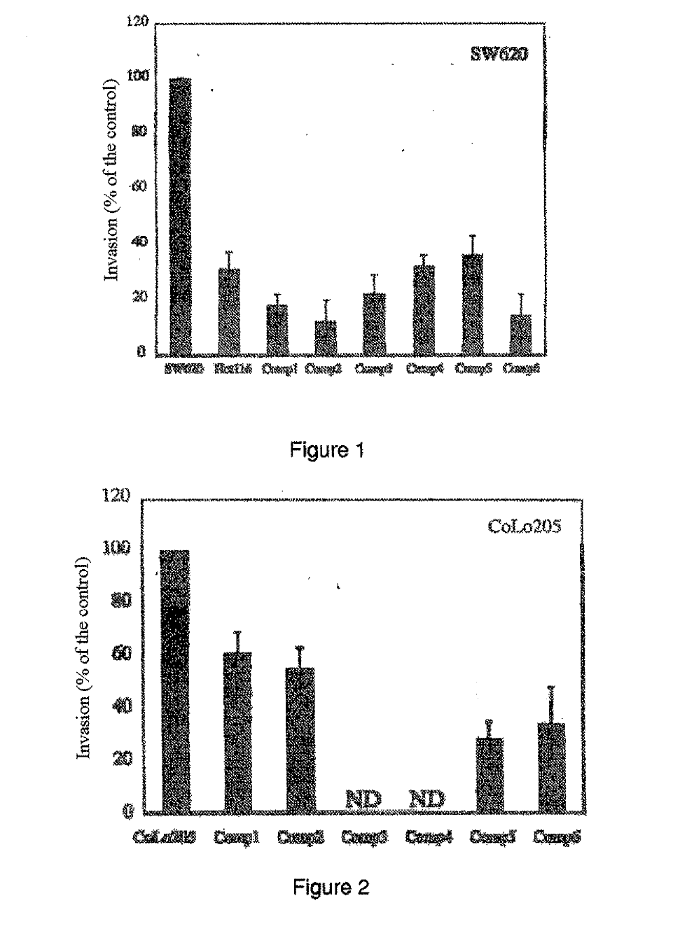

The results are shown in FIG. 1.

In FIG. 1, it is shown that all of the compounds of formula (I) to (VI) are capable of blocking the invasive properties of the cells of the metastatic colorectal cancer cell line, whereas the cells treated have an invasive activity which is similar to or less than that of the nonmetastatic colorectal cancer line HCT116.

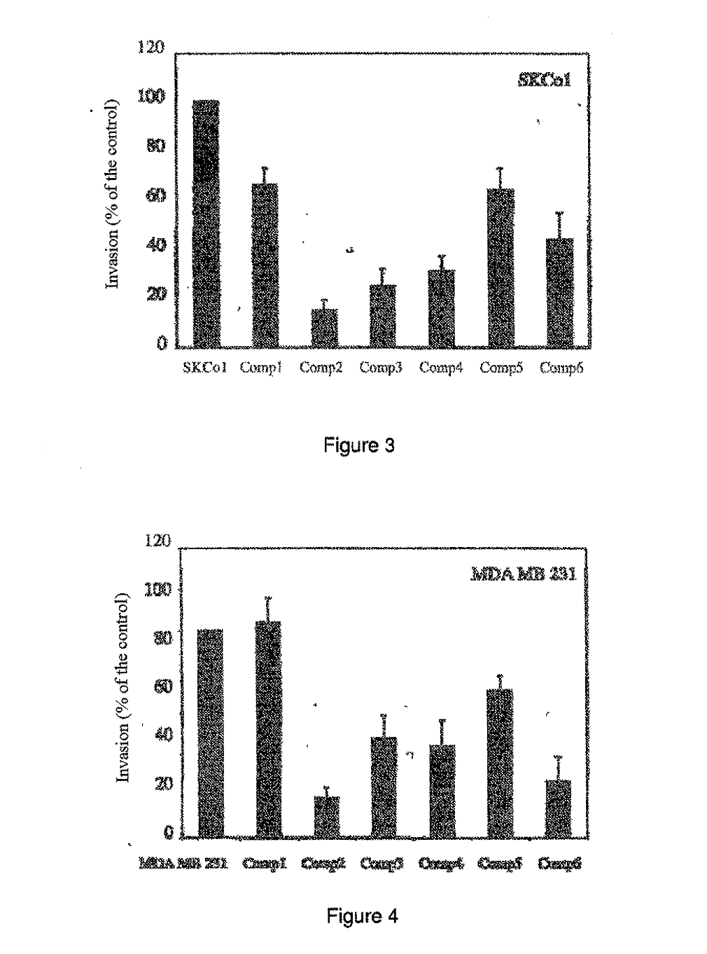

Moreover, the results of FIGS. 2, 3, and 4 show that the anti-invasive properties of each of the compounds of formula (I) to (VI) are demonstrated on various types of metastatic human cancer cells, including cells originating from a breast cancer (FIG. 4).

1-10. (canceled)

11. A method for the prevention or treatment of cancerous metastases in humans or animals consisting in administering a compound selected from an aminopeptidase inhibitor compound and an azaindole compound.

12. The method of claim 11, wherein the aminopeptidase inhibitor compound is the compound of formula (I) below:

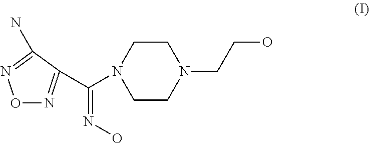

13. The method of claim 11, wherein the azaindole compound is the compound of formula (II) below: 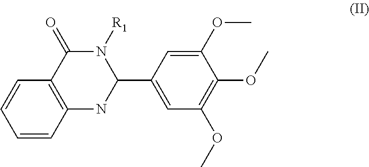
in which the group R1 is a linear or branched alkyl having from 1 to 6 carbon atoms.

14. The method of claim 11, wherein the azaindole compound is the compound of formula (III) below: 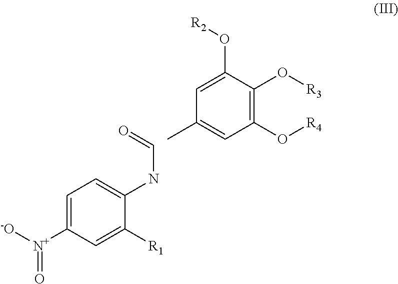
in which the groups R1, R2, R3, and R4 each represent, independently of one another, an alkyl of 1 to 6 carbon atoms.

15. The method of claim 11, wherein the azaindole compound is the compound of formula (IV) below: 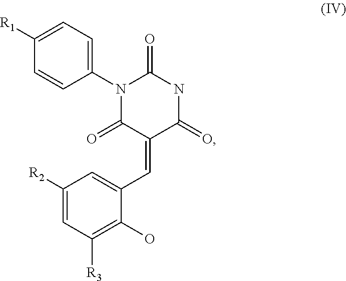
in which the groups R1, R2, and R3 each represent, independently of one another, a halogen selected from F, Cl, I, and Br.

16. The method of claim 11, wherein the azaindole compound is the compound of formula (V) below: 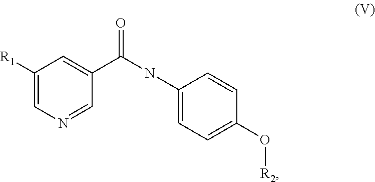
in which:
the group R1 is a halogen selected from F, Cl, I, and Br, and
the group R2 is a linear or branched alkyl having from 1 to 6 carbon atoms.

17. The method of claim 11, wherein the azaindole compound is the compound of formula (VI) below: 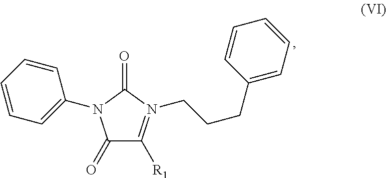
in which the group R1 is a halogen selected from F, Cl, I, and Br.

18. The method of claim 11, wherein the azaindole compound is selected from the following compounds:
methyl 4-[([3-(4-fluorophenyl)-4-oxo-2-thioxo-1,3-thiazolidin-5-ylidene)methyl)benzoate;
3,4,5-triethoxy-N-(2-methyl-4-nitrophenyl)benzamide;
5-(5-bromo-3-chloro-2-hydroxybenzylidene)-1-(4-chlorophenyl)-2,4,6-(1H,3H,5H)pyrimidinetrione;
5-bromo-N-(4-butoxyphenyl)nicotinamide; and
3-chloro-1-phenyl-4-[(2-phenylethyl)amino]-1H-pyrrole-2,5-dione.

19. The method of claim 11, wherein said medicament is intended for the prevention or treatment of epithelial cancers.

20. The method of claim 19, wherein the epithelial cancer is selected from a colorectal cancer and a breast cancer.
